(19) United States
(12) Patent Application Publication
(10) Pub. No.: US 2004/0126891 A1
(43) Pub. Date: Jul. 1, 2004

(54) Method for assaying reactive oxidants in smoke

(75) Inventors: Dejian Huang, Randolph, MA (US); Boxin Ou, Stoughton, MA (US)

Correspondence Address: Dr. Dejian Huang, 6 Thatcher Ln, Wareham, MA, 02571, US

(73) Assignee: Brunswick Laboratories, Wareham, MA

(21) Appl. No.: 10/328,666

(22) Filed: Dec. 26, 2002

(51) Int. Cl. G01N 33/00

(52) U.S. Cl. 436/127; 436/144; 436/172; 436/164; 436/161; 436/904

(57) ABSTRACT

A method of assaying the reactive oxidants present in a smoke sample, the method comprising: preparing solution including a reductant; passing smoke through the solution; detecting the concentration changes of the probe in the presence of the smoke sample over time; and calculating the concentration of reactive oxidants of the smoke sample from the concentration changes of the reductant in the presence of the smoke sample. A method of assaying the reactive oxidants present in a smoke sample, the method comprising: preparing a solid material containing a reductant; passing smoke through the solid material; detecting the concentration changes of the reductant in the presence of the smoke sample over time; and calculating the concentration of reactive oxidants of the smoke sample from the concentration changes of the reductant in the presence of the smoke sample.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISK APPENDIX

Not applicable

### BACKGROUND OF THE INVENTION

This invention relates to a method for assaying reactive oxidants in smoke. Some related references include:

Pryor, W. A.; Cigarette smoke radicals and the role of free radicals in chemical carcinogenicity. Environmental Health Perspective 105 (suppl 4): 875-882 (1997).

Hoffmann D, Hoffmann I, El-Bayoumy K. The less harmful cigarette: a controversial issue. a tribute to Ernst L. Wynder, Chem. Res. Toxicol. 14(7), 767-90 (2001).

Pilz H, Oguogho A, Chehne F, Lupattelli G, Palumbo B, Sinzinger H. Quitting cigarette smoking results in a fast improvement of in vivo oxidation injury (determined via plasma, serum and urinary isoprostane), Thromb Res 99(3):209-21 (2000).

Pryor, W. A., Cigarette smoke radicals and the role of free radicals in chemical carcinogenicity Environmental Health Perspectives, 105 (suppl 4): 875-882 (1997).

Pryor, W. A.; Tamura, M.; Church, D. F.; ESR spin trapping study of the radicals produced in NOx/olefin reactions: a mechanism for the production of the apparently long-lived radicals in gas-Phase cigarette smoke. J. Am. Chem. Soc. 106:5073-5079 (1984).

Saito, K.; Yoshioka, H.; Kazama, S.; Cutler, R. G. Release of nitric oxide from a spin trap, N-tert-butyl-alpha-phenylnitrone, under various oxidative conditions. Biological & Pharmaceutical Bulletin 21 (4): 401-404 APR (1998).

Flicker, T. M.; Green, S. A. Comparison of gas-phase free-radical populations in tobacco smoke and model systems by HPLC Environmental Health Perspectives 109 (8): 765-771 (2001).

The hazardous chemicals presented in the air are a major health threat to human being. These chemicals get into the air as a result of human or natural activity. The air pollution sources include:

(1) volatile organic chemicals (VOC) released from chemical factories and consumer products containing VOCs

(2) toxicants generated from burning organic matters including but not limited to gasoline, wood, garbage, dead animals, and tobacco.

(3) volatile chemicals derived from natural events such as forest fires, active volcanoes.

The Environmental Protection Agency (EPA) of the United States has compiled a list of identified 188 air-polluting chemicals (source: http://www.epa.gov/ttn/atw//188polls.html) ranging from benzene to inorganic elements such as mercury and cadmium. Besides the chemicals on the list, there are other unidentified toxins in the air or smoke phase of a burning matter. One group of toxins is reactive oxidants (abbreviated as ROS in this patent). Here the ROS include:

(1) free radicals, such as peroxyl, alkoxyl, nitric oxides;

(2) non-radical oxidants, such as peroxides, sulfur oxides, hypochloric acid.

The ROS exclude oxygen gas itself. Although oxygen is an benign oxidant, it does not poses a direct harm. So far we know little about the concentration and toxicological effects of the ROS in the air because we do not have a valid method to quantify them until now.

One preventable source of air pollution is cigarette smoke (abbreviated as CS in this patent). Combustion of cigarettes, like many other combustion processes, produces ROS. Numerous evidences have suggested that ROS cause unwanted oxidation of lipids, proteins and DNA. CS may also contain meta-stable non-radical oxidants (e.g. ROOH, ROONO₂, ROONO, or H₂O₂), short-lived (highly reactive) free radicals, and meta-stable free radicals (NO, NO₂, and phenolic radicals). The peroxides normally do not oxidize biomolecules directly, but will be converted to highly reactive radicals (RO, and HO) in the presence of redox active transition metals such as Fe(II). The ROS can oxidize biomolecules by hydrogen atom or electron abstraction. In general, ROS can be linked directly to CS related diseases.

ROS in CS→oxidative stress→CS related diseases

ROS are harmful species that are blamed for causing a wide range of diseases including, cancer, stress, ageing, heart, vascular, and neurogenerative diseases. In vivo, free radicals are generated as by-products of oxygen metabolism. In vitro, smokes derived from burning organic matters are long known be the source of ROS. Common organic matters include wood, cigarette, petroleum and coal-based materials.

Significant literatures reported that CS consumed through mainstream or passively from environment, induces oxidative stress. Oxidative stress has been suggested to be the cause of various diseases, including cancer, cardiovascular diseases, inflammations, and stroke. These diseases have higher incidence rate among smokers (CDC, Annual Smoking-Attributable Mortality, Years of Potential Life Lost, and Economic Costs—United States, 1995-1999, MMWR Weekly, 51(14): 300-3 (2002)). Two aspects of evidences on CS induced oxidative stress have been garnered, e.g. lowered plasma antioxidant concentrations and elevated oxidative stress biomarkers in smokers. Yang and coworkers observed that cigarette smoke induced direct DNA damage on human B-lymphoid cells. Radical scavengers, beta-naphthoflavone and coumarin, could only slightly inhibit the damage, while N-acetylcysteine reduced DNA damage (Yang, Q.; Hergenhahn, M.; Weninger, A.; Bartsch, H. Cigarette smoke induces direct damage in the human B-lymphoid cell line Raji, Carcinogenesis, 20(9): 1769-1775, (1999)). In the other study, cigarette smoke was found to inhibit catalase activity in vitro. This would enhance free radical generations due to the accumulation of H₂O₂ (Mendez-Alvarez E.; Soto-Otero, R.; Sanchez-Sellero, I.; Lamas, M. L. R. J. In vitro inhibition of catalase activity by cigarette smoke: relevance for oxidative stress J. Applied Toxicology, 18(6): 443-448 (1998)). Howard and coworkers reported 63% increase 8-hydroxy-2′-deoxyguanosine (8-oxo-2-dG, a DNA oxidation marker) concentration in the blood of subjects exposed to environmental CS (Howard, D. J.; Ota, R. B.; Briggs, L. A.; Hampton, M.; Pritsos, C. A. Environmental cigarette smoke in the workplace induces oxidative stress in employees, including increased production of 8-hydroxy-2′-deoxyguanosine, Cancer Epidemiology Biomarkers & Prevention 7(2): 141-146 (1998)). Marangon and coworker in France surveyed 459 healthy men aged 23-57 years and found that plasma vitamin C and beta-carotene concentrations were reduced in smokers compared with nonsmokers, and were inversely related to cigarette consumption (Marangon, K.; Herbeth, B.; Lecomte, E.; Paul-Dauphin, A.; Grolier, P.; Chancerelle, Y.; Artur, Y.; Siest, G.; Diet, antioxidant status, and smoke habits in French men, Am. J. Clin. Nutr. 67(2): 231-239 (1998)). In a survey of 817 adults at Nuremberg, Germany it was revealed that, for smokers, plasma concentrations of high-density lipoprotein cholesterol, triglycerides, homocysteine, cobalamin, folate, beta-carotene, and alpha-tocopherol showed a unfavorable levels (Trobs, M, Renner, T.; Scherer, G.; Heller, W. D.; Geiss, H. C.; Wolfram, G.; Haas, G. M.; Schwandt, P.; Nutrition, antioxidants, and risk factor profile of nonsmokers, passive smokers and smokers of the Prevention Education Program (PEP) in Nuremberg, Germany, Preventive Medicine 34 (6): 600-607 (2002)).

In 1993, Annals of the New York Academy of Sciences published a thematic issue on cigarette smoke. In the issue many papers were presented on smoke induced oxidative stress (Tobacco Smoking and Nutrition: Influence of Nutrition on Tobacco-Associated Health Risks. Conference proceedings. Lexington, Ky., Sep. 14-16, 1992 Ann NY Acad Sci 686:1-366 (1993)). Finally, online search on pubmed.gov database uncovers over one hundred research articles relating CS to various oxidative stress conditions. Based on these publications, it is compelling that the immediate physiological changed by CS is oxidative stress. Logically, one can envision that longtime smokers will suffer accumulative oxidative stress and CS related diseases are developed by continuous dosage of harmful ROS.

Free radicals in CS have been qualitatively studied by several researchers by electron spin resonance (ESR) technology. In 1958, Lyons and coworkers reported ESR signals from CS condensate and shortly afterwards, free radicals were detected on whole cigarette smoke (Lyons, M. J., Gibson, J. F., and Ingram, D. J. E. Free radicals in cigarette smoke. Nature 181: 1003-1004 (1958). Pryor and coworkers an ESR signal in aqueous extracts of cigarette “tar” (ACT) and assigned the chemical structures of the radical to be long-lived semiquinone based on the similarity of the ESR signals of ACT and that of an aged catechol solution (Pryor, W. A.; Cigarette smoke radicals and the role of free radicals in chemical carcinogenicity. Environmental Health Perspective 105 (suppl 4): 875-882 (1997)). The authors also applied spin trap method for identifying and quantifying gas phase radicals. In the study, phenyl-tert-butyl nitrone (PBN), 5,5-dimethyl-1-pyrroline-N-oxide (DMPO), and a-(3,5-di-tert-butyl-4-hydroxyphenyl)-N-tert-butyl nitrone (OHPBN) were used to trap the highly reactive radicals. Based on the ESR signal patterns and intensity, the authors further suggested that the R. groups include RO., carbon centered radicals, and nitric oxide and that there were estimated 5 nmol carbon-centered radicals generated in the gas phase of one 1R1 research tobacco burnt (Pryor, W. A.; Tamura, M.; Church, D. F.; ESR spin trapping study of the radicals produced in NOₓ/olefin reactions: a mechanism for the production of the apparently long-lived radicals in gas-Phase cigarette smoke. J. Am. Chem. Soc. 106:5073-5079 (1984)). No peroxyl radical (ROO.) was detected, as it is shown not reactive towards PBN by Saito and coworkers (Saito K, Yoshioka H, Kazama S, Cutler R. G. Biological & Pharmaceutical Bulletin 21 (4): 401-404 APR (1998)). ROO., a reaction product from R. and oxygen in the air, is believed to be the predominant radicals in CS. Green at Michigan Technological University detected carbon-centered radicals in CS using a spin trap, 3-amino-2,2,5,5-tetramethyl-1-pyrrolidinyloxy radical (3AP), that is specific to carbon-centered radicals. CS was passed through a column packed with glass beads coated with 3AP. The trapped non-radical amine was washed from the beads. The separated 3AP-R was further converted to fluorescent compounds by reaction with naphthalenedicarboxaldehyde (NDA) to give fluorescent adduct 3AP-R-NDA. Finally, the concentrations of 3AP-R-NDA were quantified by HPLC coupled with a fluorescent detector. Their results suggested that concentration carbon-centered radicals are ten times higher (54 nmol) in one Marlboro cigarette than that in one 1R1 cigarette (Flicker, T. M.; Green, S. A. Comparison of gas-phase free-radical populations in cigarette smoke and model systems by HPLC. Environmental Health Perspectives 109 (8): 765-771 (2001)). Kinetic analysis of the gas phase radical reactions led the authors to conclude that there were 5000 nmol radicals in the smoke of one Marlboro cigarette. Carbon-centered radicals are believed to contribute to only one percent of the total radicals.

Spin trapping coupled with ESR or HPLC did provide direct proof that there are free radicals in CS. However, it is not a practical quantitation method for radicals because spin trapping and ESR measurements involve tedious experimental procedures. In addition, this method only detect very small fraction of radicals and completely neglects non-radical ROS in CS. Despite these drawbacks, researchers in Tobacco Company have been using ESR to quantify free radicals in CS. In particular, chemists are R. J. Reynolds company trapped free radicals from heated cigarette (Eclipse™) in an PBN/benzene solution to form a stable radical species followed by detection with an electron spin resonance spectrometer (ESR). Analysis of cigarettes which include a new carbon filter and an experimental tobacco blend demonstrated a vapor phase free radical reduction on the order of 80% when compared to other equivalent tar cigarettes. Reductions observed for a cigarette, which primarily heats, rather than burns, tobacco are even greater (88-97%). It was later shown that Eclipse™ vapor phase has radical concentration of 2.22×10¹⁴ spin/cigarette. This value was 95% lower than that of research cigarette, 1R4F (4.85×10¹⁵ spin/cig) (R. J. Reynolds Company, Eclipse, A cigarette that primarily heats rather than burns the tobacco, Summary of scientific tests. (2000)). Without knowing the limitations of the ESR method one would conclude that the harmful free radicals in Eclipse™ is dramatically reduced. This conclusion, if used as a product promotion tool, would convince many smokers that Eclipse™ is less harmful.

By far the method in studying free radicals in smoke is spin trapping and electron spin resonance (esr) spectroscopy. This method suffers several disadvantages, (1), it is a selective method and thus can only detect certain radicals that are reactive to the spin trap molecules, (2) the sample collection and esr measurement are technically inefficient, tedious and difficult to validate; (3) it involves sophisticated instrumentations that are not commonly available to many researchers and public; (4) this method does not reflect non radical oxidants. In light of the need in advancing our knowledge of smoke radicals and discover the right antioxidants that can effectively quench free radicals in smoke and, consequently, reduce the hazard of smoke to human health, we designed a fluorometric assay that overcome the disadvantages of the spin-trapping method. This fluorometric method takes advantage of reaction between free radicals and fluorescent molecular probes, including, but not limited to, dihydrorhodamine-123 (DHR-123), DHR-6G, redox sensor™, and hydroethidium. The reaction is effected by hydrogen atom transfer followed by an electron transfer from DHR-123 to the free radicals, including carbon centered (R.), oxygen-centered (RO., ROO.), and nitrogen centered free radicals (NO, NO₂). Other non-radicals oxidants may also oxidize the probes and be quantified. Upon contact with these oxidants, the reductive probe was oxidized and degree of oxidation is measured by fluorescent intensity changes. Therefore, this approach can give a quantitation of global reactive oxidants in smoke (FIG. 5).

### BRIEF SUMMARY OF THE INVENTION

It is an object of this invention to provide a method of quantifying reactive oxidants in a smoke sample. The smoke sample is generated from a burning organic matter selected from, but not limited to, wood, tobacco, cigar, cigarette, gasoline, diesel fuel, and coal.

It is an object of this invention to provide such a method using a reductant, which is reactive to majority of free radicals and non radical oxidants in a smoke sample.

It is a further object of this invention to provide such a method, which is a time efficient and technically unsophisticated method for assaying the reactive oxidants in smoke.

It is a further object of this invention to provide such a method, which can be used to study toxicology effects of reactive oxidants in cigarette smoke.

It is a further object of this invention to provide such a method, which is a tool in screening antioxidant activity in cigarette smoke.

This invention results from the realization that a novel method for assaying the ROS of a smoke sample, in the preferred embodiment, can be achieved by passing smoke sample through a solution which includes a fluorescent molecular probe, monitoring the fluorescence changes of the solution, and calculating the total ROS concentration based on the changes of the fluorescence intensity by the smoke.

This invention features a method of assaying total smoke ROS scavenging capacity of an antioxidant sample, the method including preparing a smoke by burning combustible materials, passing the smoke through a solution containing fluorescent probes and chemical compounds that are known radical scavengers, detecting the fluorescence intensity changes of the probe over time, and calculating the antioxidant capacity of the sample based on the fluorescence intensity changes of the probe.

### BRIEF DESCRIPTION OF THE DRAWINGS

Other objects, features and advantages will occur to those skilled in the art from the following description of a preferred embodiment and the accompanying drawings, in which:

FIG. 1 is a picture of an apparatus showing the reaction set-up associated with the reactive oxidant measurements in smoke;

FIG. 2 is a graphical representation showing the fluorescence intensity changes of a reductant, hydroethidium, when it reacts with smoke generated from a Marlboro classic cigarette;

FIG. 3 is a graphical representation showing fluorescence intensity change overtime of Fluorescein, in the presence of different concentrations of AAPH; 37° C., [Fluorescein]=7.0×10⁻⁸ mol/L.

FIG. 4 is a graphical representation of a plot of ln([AAPH]) against ln(AUC) obtained from the result in FIG. 3.

FIG. 5 is a graphical representation of a chemical reaction of reactive oxidants in smoke and a reductant.

FIG. 6 is a graphical representation of the chemical structure of Redox Sensor™ and dihydrorhodamine-123 used for assaying the ROS of smoke sample in accordance with the subject invention;

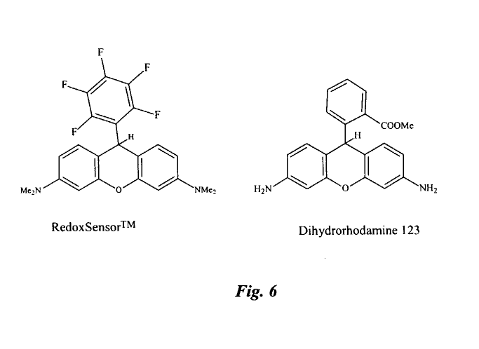

FIG. 7 is a graphical representation showing the conversion of dihydrorhodamine-123 to rhodamine-123.

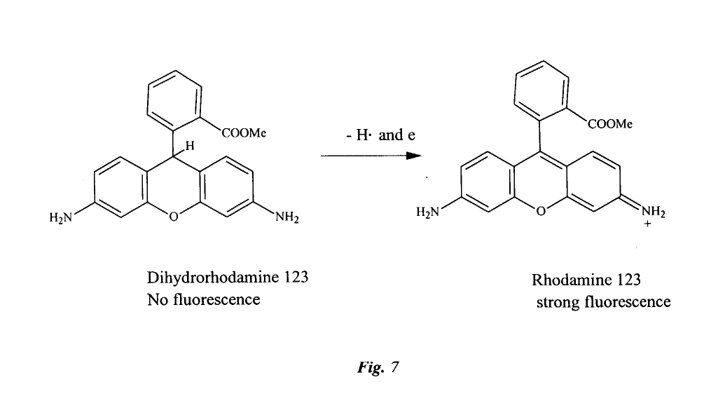

FIG. 8 is a graphical representation showing the initial fluorescence intensity changes of dihydrorhodamine-123 (DHR-123) in the presence of different concentration of AAPH. Selected kinetic curves of reaction between DHR-123 and AAPH. Data recorded on Biotek FL600A microplate fluorescence reader. Volume=175 μL. Temperature=37° C., [DHR-123]=1.43 mg/L (4.14 μM), [AAPH] (mM) and related Rᵢ (nM/min) are: ●, 4.94 (82); ▪, 1.47 (41); ▴, 0.74 (20.5).

FIG. 9 is a graphical representation showing the natural log of initial fluorescence decay rate (ln(rₒ)) and the natural log of the radical intiation rate (ln(Rᵢ)).

FIG. 10 is a graphical representation showing the initial kinetic curve of DHR-123 fluorescence changes upon reaction with tobacco smoke (Marlboro Classic). Reaction conditions: T=37° C., [DHR-123]=1.70 μg/mL. Solution volume=20 mL, solvent 75 mM phosphate buffer, gas flow rate=105 mL/min. On average, smoking one cigarette takes five min. The aliquot was collected after each cigarette smoked. The initial rate of the reaction rₒ=31.29.

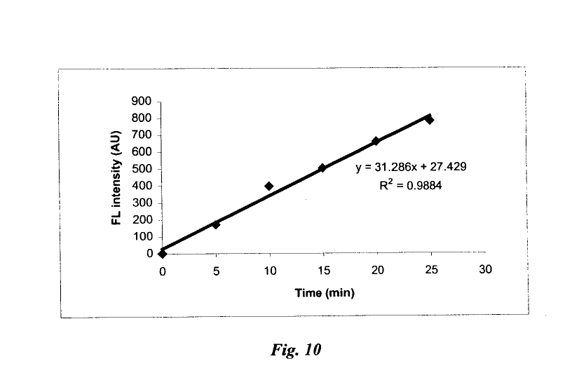

### DETAILED DESCRIPTION OF THE INVENTION

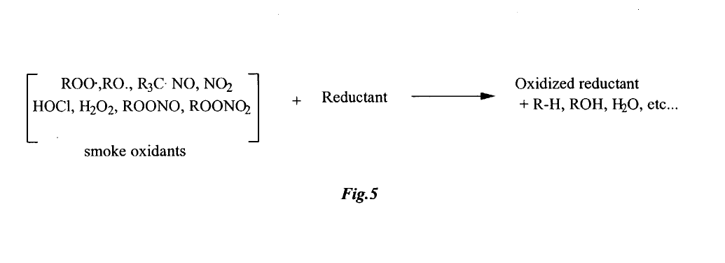

Identified ROS in smoke include non-radicals (e.g. peroxides) and radical species NO, NO₂, R., RO., and ROO.. Our purpose is to identify a reductant that is reactive towards both types of ROS (FIG. 5). There are many reductants one can choose. Ideally the reductants, or their oxidized products, should have characteristic spectroscopic properties that can be conveniently monitored with sufficient sensitivity because of potentially low ROS concentrations in CS. Yet the reductant is relatively stable towards oxygen. Feasible reductants include redox sensitive fluorescent probes. Fluorescent probes have been widely used in biochemistry to qualitatively detect reactive oxidants in biological systems (Haugland, R. P., Handbook of fluorescent probes and research chemicals 6^{th} Ed. Molecular Probe. Eugene Oreg. (1996)). The reactive oxidants detected by these probes include hydrogen peroxide (H₂O₂), hydroxyl radical (HO), hypochloric acid (HOCl), nitric oxide (NO), peroxyl radical (ROO.), peroxynitrite (ONOO⁻), and superoxide (O₂⁻). Upon reaction with these ROS, the probes are oxidized to give highly fluorescent molecules. Examples of such probes are commercially available Redox Sensor™, dihydrorhodamine (123, or 6G), and hydroethidium. In the presence of ROS, these probes are oxidized by losing an electron and a hydrogen atom to form highly fluorescent compounds, which can be detected even at very low concentrations. Our assay foundation is thus based on equation depicted on FIG. 5, where reductant is reduced fluorescent probes.

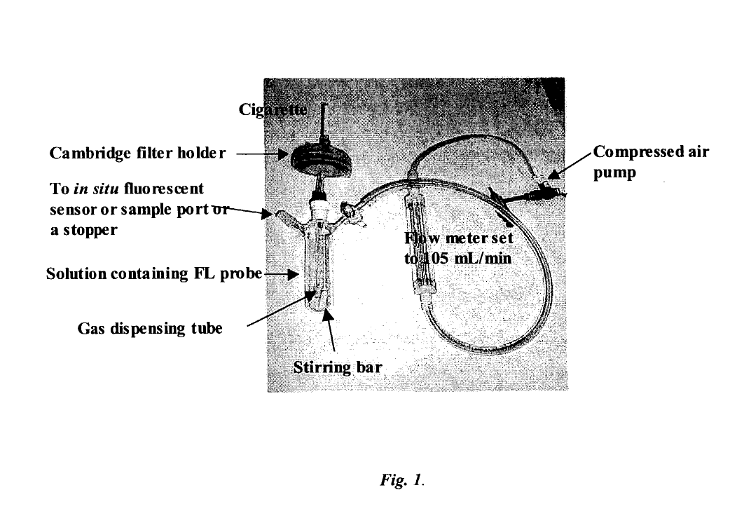

In accordance with the subject invention, smoke samples for testing are generated on a device shown in FIG. 1. Combustible materials, such as cigarette, wood and diesel oil are burnt. The generated smoke from the burning material is collected by a compressed air pump that draws the smoke to pass through a solution containing fluorescent probe in a three-neck flask. The flow rate can be varied to achieve optimal mixing of gas and the liquid. The fluorescent intensity change can either be monitored in situ by a fiber-optic fluorescent sensor, or preferably manual sampling of the reaction solution after each cigarette is smoked. The fluorescent probe solution is dissolved in a liquid, which can be any solvent or mixtures thereof including but not limited to water, phosphate buffer, dimethylsufoxide, N,N-dimethyl formamide, alcohol, acetone, acetonitrile, ethylene dichloride, arenes, and alkanes. The fiber-optic fluorescent sensor can be purchased from a commercial vendor. Plot of the fluorescence reading over time gives a kinetic curve of the fluorescence decay. The curve will be used for calculation of ROS concentrations in the smoke.

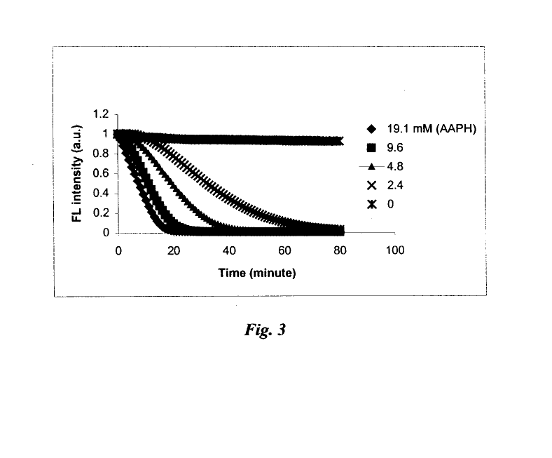

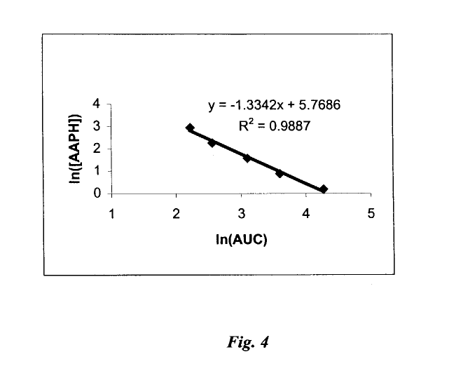

In one preferred embodiment, a solid free radical initiator, 2,2′-Azobis(2-amidino-propane) dihydrochloride (AAPH), was used as a standard. The fluorescence change curve of the probe was monitored against various AAPH concentrations (FIG. 3). The area under the fluorescence curve was plotted against the AAPH concentrations to obtain a standard calibration equation (FIG. 4). Alternatively, the initial rates of fluorescence decay were plotted against the AAPH concentrations. The plot of ln(Rᵢ) and ln(rₒ) gives linear curve that is used as a standard calibration curve for computations of free radicals generated from smoking materials.

In one preferred embodiment, commercially available cigarettes are burnt and the smoke was passed through the solution containing fluorescent probes using the setup shown in FIG. 1. The fluorescence change of the probe was monitored and the area under the kinetic curve was integrated. The AAPH equivalency of the ROS in the smoke was derived by using the standard calibration equation.

In one embodiment, commercially available cigarettes are burnt and the smoke was passed through the solution containing fluorescent probes using apparatus as shown in FIG. 1. The initial fluorescence change of the probe was monitored and the rate of the change was calculated from the kinetic curve of the fluorescence intensity over numbers of cigarettes burnt. The rate was compared with that of standard calibration curve. ROS concentration was then calculated based on the standard curve.

Taking DHR as an example, its reaction with ROS can be expressed as follows:

a ROS+b DHR→c Rhodamine+d reduced ROS (1)

The rate law is:

r=d[rhodamine]/cdt=k[ROS]^{a}[DHR]^{b} (2)

ln(r)=ln(k)+a ln[ROS]+b ln[DHR] (3)

The DHR concentration and initial rate of the reaction (rₒ) can be obtained experimentally from the fluorescence kinetic curves of the reaction. There are three unkowns in equation (3), k, a and b. To simplify the equation, we will need to use large excess of DHR so that the reaction (1) is zero order to [DHR]. This should be practical because the radical concentration might be extremely low in smoke. Therefore, under pseudo first order conditions, equation 8 is simplified:

ln(rₒ)=ln(k′)+a ln[ROS]ₒ (4)

Equation (4) is the basis for quantitation of ROS. The initial rate of the reaction can be obtained by the kinetic curves of fluorescence changes of the probe and oxidant concentrations will be calculated by equation (4) if we know the values for ln(k′) and a. Contradictorily, we need to know at least two [ROS]ₒ and their corresponding rₒ in order to calculate ln(k′) and a. To circumvent this “catch twenty-two” situation, we need to introduce a reference standard for ROS. There are two approaches for a standard. We can arbitrarily define the ROS concentration of CS generated from a reference cigarette (e.g. 1R1) to be a constant number (for example, 100) and ROS from all other CS will be compared to that of the standard cigarette. The data of this approach is easier to compare, but we would not be able to know the absolute ROS concentration of CS. Alternatively, we can use a standard oxidant as a standard. Here we assume that the reaction mechanism of reference standard is the same as the smoke ROS. Chemically, it is a reasonable assumption, because all the ROS would principally oxidize DHR through electron and/or hydrogen abstraction from DHR. We can select a commercially available peroxyl radical initiator as a standard. Commonly used azo compounds will serve this purpose. For example, AAPH produces a slow and steady influx of free radicals. The efficiency of radical generation from AAPH decomposition is 0.5 and it is not sensitive to reaction media. In other words, one molecule of AAPH generates one molecule of peroxyl radical. The rate of radical initiation (Rᵢ) for AAPH can be expressed as:

Rᵢ=2ek₁[AAPH]ₒ (5)

Where the e is efficiency of chain initiation, k₁ is decomposition rate constant of AAPH, which has been measured to be 1.27×10⁻⁷ s⁻¹ at 30° C. and 1.36×10⁻⁶ s⁻¹ at 37° in comparison with the AAPH concentrations in the experiment. The Rᵢ and the initial concentration changes of [AAPH] remain constant. Therefore, for ROS=AAPH, equation (5) can be rewritten as the follows:

ln(rₒ)=ln(k′)+a ln(Rᵢ/2ek₁) (6)

or

ln(Rᵢ)=(1/a)ln(rₒ)+ln(2ek₁/(k′)^{1/a}) (7)

Plot of ln(Rᵢ) and ln(rₒ) should yield a linear curve with slope of 1/a and intercept of ln(2ek₁/(k′)^{1/a}. The curve will serve as a reference standard curve for quantitation of ROS influx rate in CS.

Alternatively, quantitation of reactive oxidants can be accomplished by area under the curve (AUC) approach. Specifically, a fluorescent probe, preferably Fluorescein, solution reacts with oxidants and causing fluorescence intensity decay. The decay rate is proportional to the free radical generation rate. The reaction is monitored until all the reductant is consumed. FIG. 3 depicted a typical kinetic curves obtained from the reaction of Fluorescein and AAPH under different concentrations. The area under the curve (AUC) of the reaction kinetics were calculated using the following equation:

AUC=0.5+f₁/f₀+f₂/f₀+f₃/f₀+f₄/f₀+ . . . +fᵢ/f₀ (8)

Where f₀=initial fluorescence reading at 0 minute, and fᵢ is the fluorescence reading at time I (minute). Typically, equation (8) is solved and the data analyzed in an electronic spreadsheet such as Microsoft Excel or other similar products or computer programs. The plot of natural log of area under the curve and the natural log of AAPH concentration gives a linear curve depicted in FIG. 4. This linear equation of the curve serves as a standard calibration curve for calculation of the concentration for unknown samples.

### EXAMPLES

The following examples are meant to illustrate and not limit the present invention. Unless otherwise stated, all parts therein are by weight.

All solvents, Trolox, and disodium fluorescein were obtained from Aldrich (Milwaukee, Wis.). 2,2′-azobis(2-amidino-propane)dihydrochloride (AAPH) was purchased from Wako Chemicals USA (Richmond, Va.). Assays were carried out in an apparatus in FIG. 1 for gas liquid reactions. For liquid phase reactions, the data were collected on a microplate fluorescence reader (FL600A Biotek, Inc. Winooski, Vt.). Cigarettes are purchased from local retailers. Dihydrorhodamine 123, dihydrorhodamine 6G, hydroethidium is purchased from Molecular Probes, Inc. (Eugene, Oreg.).

Standard curve Ten mg DHR-123 was dissolved in 75 mM phosphate buffer to final concentration of 1.67 μg/mL. 150 μL of the solution was pipetted in A1-H1 position of a 96-well microplate. 25 μL of AAPH solution (in 75 mM phosphate buffer, pH 7.4) was added to the following wells accordingly:

| Well | A1 | B1 | C1 | D1 | E1 | F1 | G1 | H1 |
|---|---|---|---|---|---|---|---|---|
| [AAPH] (mg/mL) | 0 | 0.0414 | 0.0207 | 0.0104 | 0.0052 | 0.0026 | 0.0013 | 0.0006 |

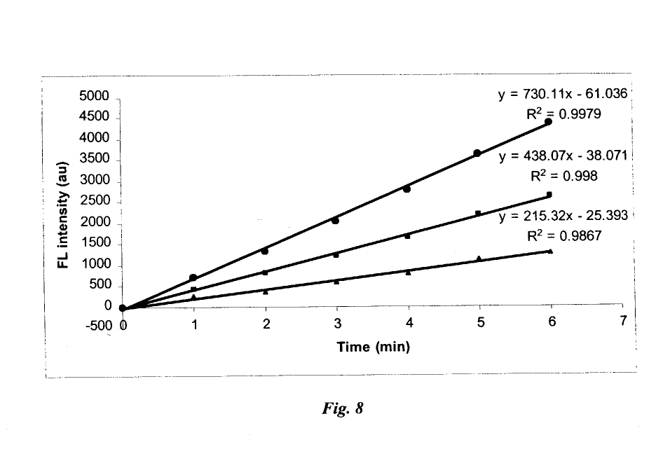

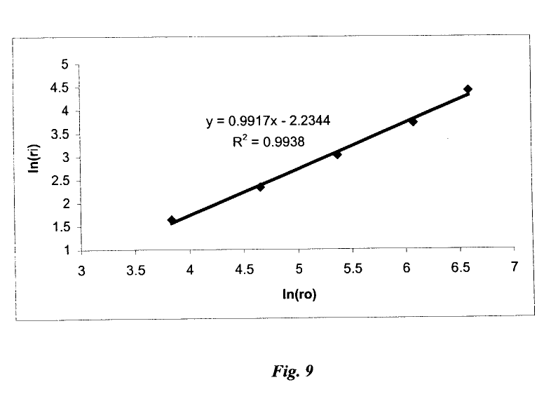

The fluorescence intensity of the wells are monitored with Biotek FL600A microplate fluorescence reader at filter of λₑₓ=485±20 nm, λₑₘ=530±25 nm. Temperature was set 37° C. The initial rate of reaction is obtained from the linearity curves (0-7 min) obtained from the plots of fluorescence vs time (min) as depicted on FIG. 8. The slope of the curve represents the rate of the reaction. The natural log plot of initial rate against the natural logs of AAPH concentrations gives a linear line as depicted on FIG. 9. The linear regression equation is:

ln(Rᵢ)=0.9917 ln(rₒ)−2.2344 (9)

Based on equation 9, we can calculate the influx rate of ROS in CS by measuring the initial rate of the reaction between the CS and the fluorescence probe.

Reaction of Fluorescein with AAPH. Fluorescein solution (8.16×10⁻⁸ mol/L, 150 μL) was placed in five different wells in a 96-well microplate. 25 μL of 0, 2.4, 4.8, 9.6, and 19.1 mM AAPH in buffer solution was added to one well. The fluorescence intensity was monitored overtime at 37° C. for eighty min. The kinetic curves were depicted on FIG. 3. The net area under the curve was calculated according to equation 8 and ln(AUC) was plotted against the natural log of ln([AAPH]) concentration. A linear line was obtained as depicted on FIG. 4. The curve can be used to serve as a standard calibration curve.

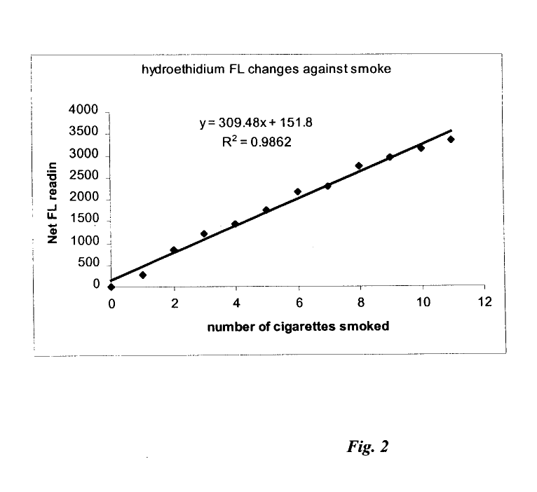

Reaction of cigarette smoke with hydroethidium. 11.0 mg hydroethidium was weighted and dissolved in 36.6 mL 2 to 1 mixture dimethyl sulfoxide (DMSO) and phosphate buffer (pH 7.4). The molar concentration of the hydroethidium is 0.3 mg/mL. 20 mL of the solution was added to a three-neck flask with a gas dispenser, an outlet to aspirator, and a sample port. Cigarette (Marlboro classic) smoke was induced to the solution at a flow rate of 105 mL/min. After each cigarette was smoked, an aliquot of the solution (10 μL) was taken and the fluorescence intensity of the aliquot was measured after dilution of 150 times. The plot of fluorescent intensity and the number of cigarette smoked was depicted on FIG. 2. Filters: excitation: 505 nm; emission 620±40 nm.

Quantitation of ROS in a Marlboro cigarette smoke We studied the reaction of a Marlboro cigarette smoke with DHR 123. The reaction apparatus is depicted on FIG. 3. No filter was used in the experiment and the results reflect total ROS in mainstream smoke. Cigarette smoke is drawn by a compressed air pump to pass through a 20 mL solution containing DHR 123 in high boiling point solvent (50% DMSO in 75 mM phosphate buffer, pH=7.4). Flow rate of the smoke is set at 105 mL/min. The CS was induced to the solution through a gas dispensing tube to enhance mixing between gas and the liquid, which was agitated magnetically on a stirring plate with a water bath set to 37° C. An aliquot was taken after each cigarette smoked and the fluorescence intensity was measured using a Biotek FL600A microplate fluorescence reader after proper dilutions. Filters used: excitation:

FIG. 5 depicts the kinetic curve of fluorescence over time caused by the smoke of Marlboro classic. Under the experimental conditions, it takes five minute to finish burning one cigarette. Apparently, the increase of fluorescence is linearly proportional to the numbers of cigarettes burnt. The initial rate of fluorescence change is equal to the slope of the curve, 31.286. Apply equation 9, we will have estimated ROS influx rate for the cigarette smoke:

ln(Rᵢ)=0.9917 ln(rₒ)−2.2344=0.9917 ln(31.286)−2.2344=1.18

Rᵢ=exp(1.1)=3.25 (nM/min)

The reaction volume is 20 mL and moles of radicals generated per minute is

20×10⁻³ (L)×3.25 (nmol/L/min)=6.50×10⁻² (nmol/min)

Therefore, amount of radicals in a Marlboro cigarette smoke per puff (35 mL) is:

35 (mL)×6.5×10⁻²/105 (mL)=2.17×10⁻² nmol

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

Other embodiments will occur to those skilled in the art and are within the following claims.

We claim:

1. A method of assaying the reactive oxidants of a smoke sample, the method comprising: preparing a solution including a reductant; passing smoke through the solution; detecting the concentration changes of the reductant in the presence of the smoke sample over time; and calculating the concentration of reactive oxidants of the smoke sample from the concentration changes of the reductant in the presence of the smoke sample;

2. The method of claim 1 in which the solution includes a high boiling-point solvent, with boiling point of no less than 50° C. at pressure of no less than 700 mmHg;

3. The method of claim 2 in which the high boiling point solvent is selected from the group consisting water, dimethyl sulfoxide, octane, N,N-dimethylformamide, t-butylnitrile;

4. The method of claim 1 in which the solution is a mixture of at least two high boiling point solvents;

5. The method of claim 1 in which the reductant is a non-fluorescent compound;

6. The method of claim 5 in which the non-fluorescent compound is selected from the group consisting: dihydrorhodamine-123, dihydrorhodamine-6G, Redox Sensor™, hydroethidium, and 2′,7′-dichlorodihydrofluorescein diacetate.

7. The method in claim 1 in which the reductant is a fluorescent compound;

8. The method in claim 7 in which the fluorescent compound is selected from the group consisting of Fluorescein, and its derivatives, BODIPY dye, rhodamine 123;

9. The method in claim 1 in which the smoke is generated from a burning biomass;

10. The method in claim 9 in which the biomass is selected from the groups consisting: tobacco, cigar, cigarette, wood, paper, dead animals, garbage, and grass;

11. The method in claim 1 in which the smoke is generated from a burning fossil fuels;

12. The method in claim 11 in which the fossil fuel is selected from the group consisting: natural gas, gasoline, diesel, coal, charcoal, and carbon;

13. The method in claim 1 in which the smoke is generated from a burning organic chemical;

14. The method in claim 13 in which the organic chemical is selected from a group consisting: alcohols, ketones, organic acids, alkanes, alkenes, alkynes, aromatic compounds, and halogenated compounds;

15. The method in claim 1 in which the concentration of the reductant is monitored by fluorescence changes of the solution overtime;

16. The method in claim 1 in which the concentration of the reductant is monitored by ultraviolet-visible spectroscopic changes overtime;

17. The method in claim 1 in which the concentration changes is monitored by a chromatographic method;

18. The method in claim 17 in which the chromatographic method is selected from a group consisting: high performance liquid chromatograph, gas chromatograph, and thin layer chromatograph;

19. The method of claim 1 in which the calculating step includes comparing the initial rate of concentration change of the reductant in the presence of a smoke sample with the initial rate of concentration change of the reductant in the presence of each standard;

20. The method of claim 19 in which each standard is an azo compound;

21. The method of claim 19 in which the azo compound is selected from a group consisting: 2,2′-azobis(2-amidino-propane)dihydrochloride (AAPH), 2,2′-Azobis[2-(5-methyl-2-imidazolin-2-yl)propane]dihydrochloride, 2,2′-Azobis[2-(2-imidazolin-2-yl)propane]dihydrochloride; 2,2′-Azobis(4-methoxy-2,4-dimethyl valeronitrile); and 2,2′-Azobis(2,4-dimethyl valeronitrile);

22. The method of claim 20 in which the concentration of each standard ranges from 0.01 μM to 1 M;

23. A method of assaying the reactive oxidants of a smoke sample, the method comprising: preparing a solid material containing a reductant; passing smoke through the solid material; detecting the concentration changes of the reductant in the presence of the smoke sample over time; and calculating the concentration of reactive oxidants of the smoke sample from the concentration changes of the reductant in the presence of the smoke sample;

24. The method in claim 23 in which the solid material is selected from a group consisting of Cambridge filter pad, filter paper, silica gel, alumina, charcoal, or cigarette filter tip;

25. The method of claim 23 in which the reductant is a non-fluorescent compound;

26. The method of claim 25 in which the non-fluorescent compound is selected from the group consisting: dihydrorhodamine-123, dihydrorhodamine-6G, Redox Sensor™, hydroethidium, and 2′,7′-dichlorodihydrofluorescein diacetate.

27. The method in claim 23 in which the reductant is a fluorescent compound;

28. The method in claim 27 in which the fluorescent compound is selected from the group consisting of Fluorescein, and its derivatives, BODIPY dye, rhodamine-123;

29. The method in claim 23 in which the smoke is generated from a burning biomass;

30. The method in claim 23 in which the biomass is selected from the group consisting: tobacco, cigar, cigarette, wood, paper, dead animals, garbage, and grass;

31. The method in claim 23 in which the smoke is generated from a burning fossil fuel;

32. The method in claim 31 in which the fossil fuel is selected from the group consisting: natural gas, gasoline, diesel, coal, charcoal, and carbon;

33. The method in claim 23 in which the smoke is generated from a burning organic chemical;

34. The method in claim 33 in which the organic chemical is selected from a group consisting: alcohols, ketones, organic acids, alkanes, alkenes, alkynes, aromatic compounds, and halogenated compounds;

35. The method in claim 23 in which the concentration of the reductant is monitored by fluorescence changes of the solid material overtime;

36. The method in claim 23 in which the concentration of the reductant is monitored by ultraviolet-visible spectroscopic changes;

37. The method in claim 23 in which the concentration changes is monitored by a chromatographic method;

38. The method in claim 37 in which the chromatographic method is selected from a group consisting: high performance liquid chromatograph, gas chromatograph, thin layer chromatograph;

39. The method of claim 23 in which the calculating step includes comparing the initial rate of concentration change of the reductant in the presence of a smoke sample with the initial rate of concentration change of the reductant in the presence of each standard;

40. The method of claim 39 in which each standard is an azo compound;

41. The method of claim 39 in which the azo compound is selected from a group consisting: 2,2′-azobis(2-amidino-propane)dihydrochloride (AAPH), 2,2′-Azobis[2-(5-methyl-2-imidazolin-2-yl)propane]dihydrochloride, 2,2′-Azobis[2-(2-imidazolin-2-yl)propane]dihydrochloride; 2,2′-Azobis(4-methoxy-2,4-dimethyl valeronitrile); and 2,2′-Azobis(2,4-dimethyl valeronitrile);

42. The method in claim 39 in which each standard is either nitric oxide, or nitric dioxide, or the mixtures thereof.
